(19) United States
(12) Patent Application Publication
(10) Pub. No.: US 2007/0059261 A1
(43) Pub. Date: Mar. 15, 2007

(54) Antimycotic nail polish formulations comprising substituted 2-aminothiazoles as an active substance

(76) Inventor: Susanne Lang-Fugmann, Ratingen (DE)

Correspondence Address: JONES DAY, 51 Louisiana Avenue N.W., Washington, DC, 20001-2113, US

(21) Appl. No.: 10/554,703

(22) PCT Filed: Jan. 24, 2004
(86) PCT No.: PCT/EP04/00597

§ 371 Date: Oct. 9, 2006

(30) Foreign Application Priority Data
Sep. 11, 2003 (DE) 103 41 944.6

(51) Int. Cl. A61K 31/506; A61K 8/49

(52) U.S. Cl. 424/61; 514/275

(57) ABSTRACT

Antimycotic nail polish formulations for the treatment of various fungal infestations are disclosed in this application. The formulations comprise substituted 2-aminothiazole compounds disclosed herein.

### FIELD OF THE INVENTION

This invention relates to antimycotic nail polish formulations for the treatment of onychomycoses and other fungal infestations affecting finger- or toenails. More particularly, the invention relates to antimycotically active nail polish formulations, which are applied to nails to form a film from which the antimycotic agent is released and can penetrate the nail. This method can be used for treating or preventing fungal infestations of human or animal nails.

### BACKGROUND OF THE INVENTION

Fungal infections of the nails, commonly referred to as onychomycoses, are most commonly caused by dermatophytes, but can also be caused by moulds and yeasts. Mixed infections also occur. Onychomycoses therefore include dermatophyte infections of the nail plate and nails as well as infections caused by other fungi, including moulds and yeasts. Furthermore, onychomycoses can serve, for example, as a reservoir for dermatophytes and thereby lead to treatment failure of tinea pedis and recurrence of tinea pedis.

Most commonly, onychomycoses are caused by Trichophyton rubrum, T. mentagrophytes and Epidermophyton floccosum. Onychomycoses due to non-dermatophytes are usually caused by Candida species.

Onychomycoses cause thickening and discoloration of the nail and can even cause loss or destruction of the nail, furthermore they can cause pain, insufficient circulation, problems when walking and other undesirable effects.

Treatment of onychomycoses in the past has included removal of the infected portion of the nail or removal of the entire nail. This form of treatment, however, may lead to permanent damage to the nail. Moreover, the nail may re-grow in a misshapen form. In addition, there is no guarantee that the onychomycosis is cured completely by nail removal.

Instead of nail removal, onychomycosis can also be treated with a variety of antimycotic agents. The antimycotic agents can be administered, for example, orally. However, this kind of treatment stresses the whole body, and only a small amount of the antimycotically active substance reaches the nail via the nail matrix. Moreover, oral administration is disadvantageous in that such administration requires a treatment duration of at least 12 weeks for toenails and about 6 to 8 weeks for fingernails. Such a long treatment duration causes high costs for the treatment. Also, patient compliance is markedly reduced. In addition, oral treatment increases the risk of side effects such as, for example, gastrointestinal irritation, nausea, adverse drug-to-drug interactions, drug-induced rashes, and other undesirable side effects. Moreover, variable rates of absorption and metabolism make oral treatment of onychomycoses more difficult.

Another method of treating onychomycoses involves the topical application of a pharmaceutical formulation comprising an antimycotic agent. It is known to treat onychomycoses with nail polish formulations comprising an antimycotic agent.

Nail polish formulations for the treatment of onychomycoses and similar fungal infections affecting nails, including finger- and toenails, of humans but also animals, are known.

Some examples are described in the patent literature. In this context, the following can be mentioned:

U.S. Pat. No. 4,957,730 (1-hydroxy-2-pyridone in water-insoluble film-former);

U.S. Pat. No. 5,120,530 (amorolfine in quaternary ammonium acrylate copolymer);

U.S. Pat. No. 5,264,206 (tioconazole, econazole, oxiconazole, miconazole, tolnaftate, naftifine hydrochloride, in water-insoluble film-formers);

U.S. Pat. No. 5,346,692 (with urea and dibutyl phthalate as plasticizer);

U.S. Pat. No. 5,487,776 (griseofulvin in a colloidal suspension).

Further patents with other examples are, for example, Nos. 4,636,520; 5,002,938; 5,110,809; 5,219,877; 5,391,367; 5,464,610; 5,696,105; 5,814,305; 6,224,887; 6,495,124 and WO No. 01/60325.

The effectiveness of nail polishes comprising the antimycotic agent amorolfine is described by Jean-Paul L. Marty, J. of the European Academy of Dermatology and Venereology, 4 (Suppl. 1), 17-21 (1995). The author describes that the film-forming solution of the nail polish consist principally of active agent and a volatile solvent (ethanol, ethyl-, butyl-, methyl-acetate, methylene chloride, methyl ethyl ketone, isopropanol) and a water-insoluble polymer (methacrylic acid copolymer, vinyl polymers), which form a continuous film following evaporation of the solvent. Plasticizers (triacetin, dibutyl phthalate) impart sufficient mechanical flexibility to the nail polish to prevent flaking and abrasion of the lacquer.

A review of the structure and characteristics of the nail and a discussion of the permeability of the nail plate to various agents and the permeability to alcohols is provided by K. A. Walter and G. L. Flynn: “Permeability characteristics of the human nail plate”, Int. J. of Cosmetic Science, 5, 231 to 246 (1983).

More recently, antimycotically active nail polish formulations have been developed, which contain 2-n-nonly-1,3-dioxolane or related dioxanes and acetal compounds as skin and nail permeation enhancers. It has also been discovered that these permeation enhancers function as plasticizers for film-forming polymers. Thus nail polish formulations, comprising dioxolanes, dioxanes and acetals as permeation enhancers, can be formulated without additional plasticizer. The films prepared from these formulations remain transparent, hard and water-resistant, they adhere well to nails and moreover release a sufficient amount of antimycotic agent to the nail. Such antimycotically active nail polish formulations are claimed in WO 99/39680.

From U.S. Pat. Nos. 5,914,322 and 5,962,433 it is known that hyaluronic acid can be used as permeation enhancer. Moreover, effective antimycotic nail polish formulations are described, in which hyaluronate lyases which may be obtained from different microbial sources are used as permeation enhancer (WO 00/38732).

The antimycotic agents used in the above-described nail polish formulations, however, all have an insufficient effect for the treatment of onychomycoses, because the agents are not sufficiently effective against the pathogens causing onychomycoses. Due to their fungicidal effect on growing and resting germs, the antimycotically active substituted 2-aminothiazoles allow an effective and successful treatment of onychomycoses when applied in the form of a nail polish formulations. Such nail polish formulations with substituted 2-aminothiazoles as active agent are an object of the present invention.

### DETAILED DESCRIPTION OF THE INVENTION

An object of the present invention is a composition comprising (A) a pharmaceutically active compound from the class of antimycotically active substituted 2-aminothiazoles and (B) a nail polish formulation of this compound, comprising a permeation enhancer.

The pharmaceutically active compounds from the antimycotically active substituted 2-aminothiazoles are described in U.S. Pat. Nos. 4,956,370 and 5,104,879.

The substituted 2-aminothiazoles have the general formula (I) 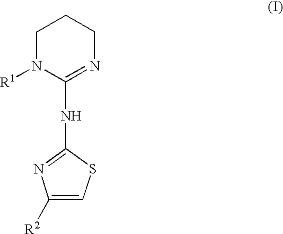
wherein

R¹ represents hydrogen or alkyl, and

R² represents a substituent of the formula 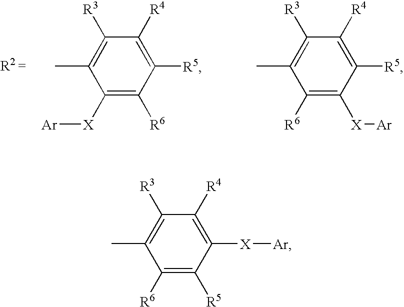
wherein

R³, R⁴, R⁵ and R⁶ independently from each other represent hydrogen, halogen, nitro, alkyl, alkoxy, alkoxy-carbonyl, dialkylamino, alkylthio, alkylsulphinyl, alkylsulphonyl, halogenoalkyl, halogenoalkoxy, halogenoalkylthio, halogenoalkylsulphinyl or halogenoalkylsulphonyl,

X represents oxygen, sulphur, sulphinyl or sulphonyl, and

Ar represents an optionally substituted aryl moiety,

and their physiologically acceptable acid addition salts.

The compounds of formula (I) are in equilibrium with the tautomeric compounds of formulae (Ia) and (Ib) 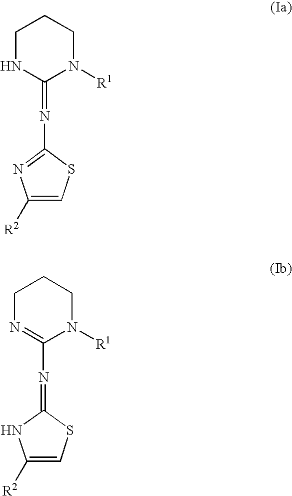
wherein R¹ and R² in each case have the above-mentioned meanings.

Especially effective is the compound of formula (I) or (Ia) or (Ib) with R¹=H and R²=

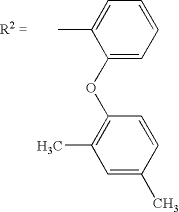

This active ingredient is called Abafungin (according to the British Pharmacopoeia).

The nail polish systems, in which Abafungin can be used, correspond to previously described systems (see, for example, Poucher's Perfumes, Cosmetics and Soaps, ed. H. Butler, Kluwer Academic Publishers, Dordrecht, Boston, London 2000, 330-343; Sudaxshina Murdan, Int. J. Pharmaceutics, 236 (2002) 1-26), the above-mentioned patents and the literature mentioned in these publications and patents.

The nail polish systems should be easy to use, harden quickly, dry quickly, be waterproof, adhere well, be elastic and resistant to flaking and abrasion. The raw materials employed may not be toxic and must be dermatologically innocuous. The main constituents of such nail polish systems are a film-former, a polymer, a plasticizer and solvents. Moreover, skin or nail permeation enhancers may be used in the nail polish formulation. As mentioned above, it has been described in some inventions that there are enhancers that also function as plasticizers for the film-forming polymer.

The present invention provides an important advance over the prior art, because onychomycoses and other fungal infections, bacterial infections and inflammations including psoriasis can be treated successfully with the nail polish formulation of the invention, comprising an active agent from the class of antimycotically active substituted 2-aminothiazoles.

### DESCRIPTION OF THE INVENTION

The pharmaceutical formulation of the present invention comprises a pharmaceutically effective amount of an antimycotically active compound from the class of substituted 2-aminothiazoles (U.S. Pat. Nos. 4,956,370 and 5,104,879). In particular, the effective pharmaceutical formulation comprises Abafungin as active agent. These nail polish formulations with substituted 2-aminothiazoles as active agent can be used therapeutically or prophylactically. The substituted 2-aminothiazoles can also be used in combination with an anti-inflammatory agent and/or another antimicrobial agent such as, for example, an antibacterial or antiviral agent.

The concentration of the substituted 2-aminothiazole, especially Abafungin, should not exceed the maximum amount that remains soluble in the pharmaceutical formulation. Most application forms comprise the pharmaceutically active substance from the class of substituted 2-aminothiazoles in an amount of 0.05% to 20% (percentages given refer to weight percent relative to the total weight of the pharmaceutical formulation). Preferably the amount of the compound is 1% to 12% (weight percent), more preferably the amount is between 1% and 10% (weight percent), and most preferably the amount is between 1.5% to 8% (weight percent).

The pharmaceutical formulation may also contain a membrane-compatible permeation enhancer which is capable of accelerating the amount of pharmaceutically active agent, which passes through the nail and/or a membrane, i.e. a layer of body tissue such as skin. The term “membrane-compatible permeation enhancer” means in this context a compound, which facilitates the penetration of the pharmaceutically active compound into the nail or skin without damage.

It is known that there are permeation enhancers which function by mechanisms such as, for example, hydrolysis, keratolysis, denaturation or other mechanisms which damage the nail and/or the skin. Examples of such permeation enhancers are urea, sulfhydryl group-containing amino acids, alkyl sulfoxides and related compounds. Such enhancers function by breaking down the structure of the nail or the skin, such that the pharmaceutically active agent can penetrate undesirably to deeper layers of the skin or even pass completely though the skin layers, thereby causing undesirable side effects.

It must be possible to use the membrane-compatible permeation enhancer safely in the treatment of infections of skin and nails without causing damage to nails or skin.

In principle any membrane-compatible permeation enhancer or mixture of permeation enhancers can be used. Preferred membrane-compatible permeation enhancers are lipophilic, such that they render membranes more permeable by embedding themselves within the membrane. Examples of such lipophilic permeation enhancers are alkylesters such as, for example, isopropyl myristate and myristyl myristate as well as macrocyclic enhancers.

The membrane-compatible permeation enhancers are used in the pharmaceutical formulation in an amount that accelerates the permeation of the pharmaceutically active agent into the nail and skin. The effective amount is known to the skilled person or can be determined easily and simply by the skilled person. In general, the amount of membrane-compatible permeation enhancer ranges from 1% to 25% (weight percent relative to the total weight of the pharmaceutical formulation).

The pharmaceutically active formulation may also comprise a polymeric film-forming agent or mixtures of film-forming agents. Essentially, any polymer can be used that is capable of forming a film from which the pharmaceutically active agent can be released into the nail or the bed of the nail or the membrane, for example, the skin. For example, occlusive and semi-occlusive polymers which are known for use in transdermal drug delivery can be used. Since the primary objective is drug release, even those polymers may be used that are otherwise rejected for cosmetic applications due to non-ideal cosmetic properties.

Examples of suitable film-forming polymers are polymers of acrylic acid, acrylic acid esters and copolymers thereof; polymers of methacrylic acid, methacrylic acid esters and copolymers thereof; polymers of vinyl acetate and copolymers thereof with acrylic acid and acrylic acid esters; copolymers of methyl vinyl ether with maleic acid, maleic acid alkyl esters and combinations thereof; copolymers of vinyl pyrrolidone with styrene; poly(vinyl butyrate); polymeric cellulose derivates such as cellulose acetate phthalate, cellulose butyl acetate, cellulose acetyl propionate, cellulose nitrate, cellulose sulfate, ethyl cellulose and cellulose acetate; terpolymers of vinyl acetate with butyl maleate and isobornyl acetate; terpolymers of vinyl caprolactam with vinyl pyrrolidone and dimethylamino ethyl methacrylate. The film-forming agents may be used as solids, for example, in powder form. Additionally the application may be realised in latex form.

Preferred film-forming agents are acrylic acid esters comprising quaternary ammonium groups, methacrylic acid ester copolymers known as ammonio methacrylate copolymers such as, for example, ethyl acrylate-[2-methacryloyloxy) ethyl]trimethylammonium chloride-methyl methacrylate copolymer, and substituted copolymers of alkylated poly(vinyl pyrrolidones). These polymeric film-forming agents are preferred, because they show superior adhesive properties, are water-resistant and exhibit a particular hardness. Particularly preferred are polymeric film-forming agents that have been registered with regulatory agencies for pharmaceutical use and that are listed in, for example, the European and US Pharmacopoeias as well as the Japanese Pharmaceutical Excipients Compendia.

Suitable film-forming polymers are commercially available such as, for example, the acrylic acid copolymers sold by the National Starch Company under the trade names DERMACRYL®, e.g. DERMACRYL 79®, DERMACRYL LT®; the amine or quaternary ammonium group containing acrylic acid copolymers sold by Röhm under the trade names EUDRAGIT®, e.g. EUDRAGITs ES, RS®, RL®; the methyl vinyl ether copolymers sold by the ISP Corporation under the trade names GANTREZ®, e.g. GANTREZ ES-3351®, GANTREZ ES-425®, ES-435®; and also the quaternary ammonium acrylic acid copolymers sold by the National Starch Company under the trade names AMPHOMER®, e.g. AMPHOMER LV-71®.

The polymeric film-forming agent is used in an amount that allows for the formation of an adherent polymeric film on the nail or the membrane, i.e. the skin. The amounts can be determined easily for any particular application by the skilled person.

Generally, for most applications, the polymeric film-forming agents are used in an amount of from 0.1% to 35%, preferably from 5% to 35%, and typically, and most preferably, in an amount of from 10% to 25% (all in weight percent relative to the total weight of the formulation).

The pharmaceutical formulation may also comprise a solvent to dissolve solid ingredients. Generally, any solvent or combination of solvents may be used. Examples of such solvents are alcohols, esters, ethers, aromatic hydrocarbons, aldehydes, ketones, mono-, di- and tri-glycerides, and the like.

The solvents which may be used in the present invention are not limited, but should be selected from the group of usually physiologically safe organic solvents for lacquer formulations. It is important that the pharmaceutical activity of the pharmaceutically active agent used is not influenced, that the lacquer can be applied easily, and that the solvent is readily volatile to accomplish acceptable drying times.

Usually the following solvents are used: ethanol, ethyl acetate, butyl acetate, isopropanol, acetone, methyl ethyl ketone, triacetin, tripropionin, diethylene glycol monoethyl ether, and isopropyl acetate, and mixtures of two or more of the aforementioned solvents.

Ethanol, ethyl acetate, butyl acetate, isopropanol, methyl ethyl ketone and acetone are especially preferred solvents, since they evaporate quickly after application and the lacquer dries quickly.

The solvent has to be present in the pharmaceutical formulation in such an amount that all ingredients are dissolved, but without the drying time being unsatisfactorily long and without influencing the lacquer properties negatively. Generally, the solvent is used in an amount of from 30% to 80% (by weight), more preferably, in an amount of from 40% to 70% (by weight).

During preparation of the pharmaceutical lacquer formulations various factors must be considered regarding the choice of the penetration enhancer, the film-forming agent and the solvent. Preferred compositions must be able to form a solid film with relatively high concentrations of pharmaceutically active agent and permeation enhancer at the interface between the solid film and the surface on which the film is formed, for example, the nail.

It is noted that the term “fugacity” is used to refer to the measure of the escaping tendency of a solute from a solution and that the fugacity of a solute follows Henry's Law for ideal states.

Various methods can be used to increase the fugacity of the pharmaceutically active agent and the permeation enhancer. These methods provide for an increase of the concentration of both ingredients at the interface.

Preferably, in relation to antimycotically active substituted 2-aminothiazoles used in accordance with the present invention which have a basic character, polymeric film-forming agents can be used which are also basic. Such basic compounds tend to repel each other due to charge distributions or polarities. Thereby the fugacity of pharmaceutically active agent from the group of 2-aminothiazoles is increased. Examples of polymeric film-forming agents with basic functionalities or basic structure elements are, for example, acrylate copolymers with dimethyl amino functionalities.

Another method for increasing the fugacity of the pharmaceutical agent from the class of substituted 2-aminothiazoles comprises adding a basic compound such as, for example, triethanolamine to the solution.

The following is a description of a method for increasing the fugacity of the permeation enhancer. The method comprises the preparation of a pharmaceutical formulation, in which the permeation enhancer is dissolved in a volatile solvent. The solvent has a vapour pressure that enable the solvent to evaporate within 5 minutes from application. The pharmaceutical formulation further comprises a less volatile solvent as cosolvent, in which the permeation enhancer only has a limited solubility of a maximum of 5% (by weight). This means that the solvent evaporates faster relative to the cosolvent and the cosolvent remains longer in the formulation. In such a case the fugacity of the permeation enhancer increases. A preferred suitable cosolvent is propylene glycol.

A further example of a method for increasing the fugacity of the pharmaceutical compound from the compound class of substituted 2-aminothiazoles, which have a low solubility in water, as well as of a permeation enhancer, which has a higher solubility in water than the substituted 2-aminothiazoles, comprises the addition of water to the formulation in an amount of up to 20% (by weight), preferably from 1% to 10% (by weight), and most preferably from 3% to 7% (by weight).

The foregoing descriptions provide examples of methods which may be employed to optimise the release of the pharmaceutical agent from the compound class of substituted 2-aminothiazoles and of the permeation enhancers.

One or more plasticizers may be used in the pharmaceutical formulation according to this invention to guarantee the desired properties of the polymeric film which is to be formed. The selection of the plasticizer and the applied amount should take into account whether the permeation enhancer to be used in the formulation has plasticizing properties itself. Examples of plasticizers are propylene glycol, diethylene glycol monoethyl ether, propylene glycol monopropyl ether, polyethylene and poly(propylene glycol), triacetin, tripropionin, castor oil, camphor, phthalates, particularly dibutyl phthalate and diethyl phthalate, benzyl alcohol, phenyl ethyl alcohol and N-methyl-2-pyrrolidone, and a mixture of two or more of the aforementioned plasticizers.

According to the state of the art, the plasticizer should match the polymeric film-forming agent to be used in the pharmaceutical formulation.

Preferably, propylene glycol, diethylene glycol monoethyl ether, polyethylene and poly(propylene glycol), 1,2,3-propanetriol triacetate (triacetin), and tripropionin, and a mixture of two or more of the aforementioned plasticizers are used in the pharmaceutical formulation of the present invention. Propylene glycol is among the preferred plasticizers.

The plasticizer is used in an amount suitable to provide the desired properties to the polymeric film that is formed from the pharmaceutical formulation of the present invention. Generally the amount of plasticizer is 1% to 25% (by weight), preferably 1% to 10% (by weight).

The pharmaceutical formulation may further comprise other ingredients recognised in the art in amounts known to the skilled person. This includes colouring agents such as, for example, a dye, colouring pigment, colouring particle, lustrous dye or pigments such as, for example, titanium dioxide and the like. Other components include colloid stabilizers, UV-stabilizers, antibacterial or bacteriostatic compounds such as, for example, antimicrobial quaternary ammonium agents, for example, cetyl pyridinium chloride and benzalkonium chloride, anti-oxidants, for example, BHA, BHT, parabens, vitamin E and its derivatives, antimicrobial chelating agents, for example, EDTA and citric acid, and neutralizing agents, for example, triethylamine, triethanolamine, 2-methyl-2-amino-1-propanol, citric acid and sorbic acid.

The pharmaceutical formulations of the present invention can be applied to the membrane or the nail or the membrane, i.e. the skin, by any method capable of forming a film. The film can be prepared by applying several layers of the pharmaceutical formulation. One or more layers may be applied after the previous layer has dried. Periodic renewal of the film may be required to provide the desired drug dosage continuously.

One of the most important features of the pharmaceutical formulation of the present invention is that all volatile and non-volatile ingredients are compatible with each other and solutions can be formed which are stable over a wide temperature range above and below room temperature such as, for example, temperatures within a temperature range of from −15 to +100° C., i.e. within this temperature range phase separation must not occur.

Another characteristic feature of the present invention is that the films formed upon evaporation of the solvents and other volatile ingredients have strongly adherent properties and are water-resistant, i.e. that they withstand in particular repeated normal washing with soapy water for at least one day, but generally up to 5 or more days, and continue to adhere.

As already mentioned the present invention provides an important advance over the prior art, because onychomycoses or other fungal infections as well as bacterial infections and inflammations, including psoriasis, can be treated with the nail polish formulation of the invention.

The nail polish formulations described in the present invention can be used not only in the therapy of diseases, but also in the prophylaxis for healthy subjects who are at risk of being exposed to mycotic or other infections.

### EXAMPLES

The following examples 1 to 7 illustrate a number of pharmaceutical formulations typical for the present invention which comprise as antimycotic agent substituted 2-aminothiazoles. The concentrations of the ingredients are given in percent by weight relative to the total weight of the pharmaceutical formulation.

The polymeric film-forming agents are dissolved in powder form or as pellets in the solvent or mixtures of solvents under mechanical stirring at room temperature. After preparation of the solution, the substituted 2-aminothiazole and the permeation enhancer are each added with stirring. When everything is dissolved, the plasticizer and optionally water are added. The entire mixture is then stirred until homogeneous.

### Example No. 1

| | wt % |
|---|---|
| Abafungin | 2% |
| Eudragit RL ® 100 powder | 15% |
| Oxacyclohexadecan-2-one (Firmenich) (permeation enhancer) | 15% |
| Propylene glycol | 5% |
| Ethanol | 60% |
| Water | 3% |

Eight patients with onychomycoses of the toenails were treated once daily in the morning with approximately 20-35 mg of the nail polish formulation of Example No. 1. The treatment was repeated each day for seven days. On the seventh day, the nail lacquer of each treatment cycle was removed by isopropyl alcohol. Thereafter the treatment cycle was repeated for 100 days. After 45 days, substantial improvements of the symptoms were observed in all cases. Complete cures were achieved in all cases within 100 to 150 days of the beginning of the treatment.

### Example No. 2

| | wt % |
|---|---|
| Abafungin | 1.5% |
| Eudragit RL ® PO pellets | 15% |
| Oxacyclohexadecan-2-one (Firmenich) (permeation enhancer) | 15% |
| Propylene glycol | 5% |
| Ethanol | 60.5% |
| Water | 3% |

### Example No. 3

| | wt % |
|---|---|
| Abafungin | 4% |
| Eudragit RL ® 100 powder | 15% |
| Oxacyclohexadecan-2-one (Firmenich) (permeation enhancer) | 15% |
| Propylene glycol | 5% |
| Ethanol | 58% |
| Water | 3% |

### Example No. 4

| | wt % |
|---|---|
| Abafungin | 4% |
| Propylene glycol | 5% |
| Hyaluronate lyase (2500 IU/cm³) | 1.3% |
| Sodium edetate | 0.1% |
| Trometamol | 0.25% |
| Polyacrylic acid | 0.5% |
| Potassium sorbate | 0.1% |
| Water | ad 100.00% |

### Example No. 5

| | wt % |
|---|---|
| Abafungin | 2% |
| Hyaluronate lyase (2500 IU/cm³) | 1.3% |
| Non-ionic hydrophilic ointment (DAB) | ad 100% |

### Example No. 6

| | wt % |
|---|---|
| Abafungin | 5% |
| Pentadecalactone | 10% |
| Eudragit RL ® 100 powder | 25% |
| Isopropanol | 60% |

### Example No. 7

| | wt % |
|---|---|
| Abafungin | 3% |
| Pentadecalactone | 15% |
| Ethanol | 82% |

1. A pharmaceutical, antimycotically active nail lacquer for the treatment or prophylaxis of fungal infections of nails and skin, comprising an optionally substituted 2-aminothiazole of formula (I) 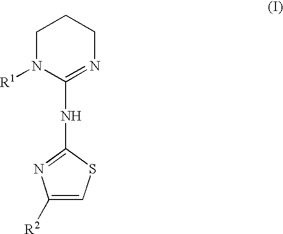
wherein
R¹ represents hydrogen or alkyl, and
R² represents a substituent of the formula 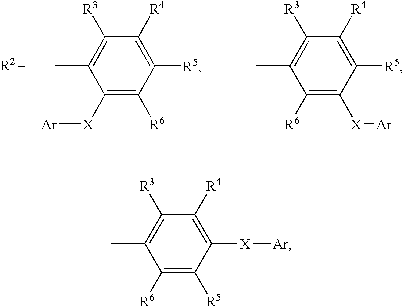
wherein
R³, R⁴, R⁵ and R⁶ independently from each other represent hydrogen, halogen, nitro, alkyl, alkoxy, alkoxy-carbonyl, dialkylamino, alkylthio, alkylsulphinyl, alkylsulphonyl, halogenoalkyl, halogenoalkoxy, halogenoalkylthio, halogenoalkylsulphinyl or halogenoalkylsulphonyl,
X represents oxygen, sulphur, sulphinyl or sulphonyl, and
Ar represents an optionally substituted aryl moiety, and
their physiologically acceptable acid addition salts,
wherein the compounds of formula (I) are in equilibrium with the tautomeric compounds of formulae (Ia) and (Ib) 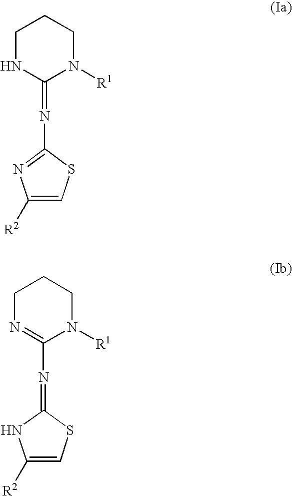
wherein R¹ and R² in each case have the abovementioned meanings.

2. A pharmaceutical, antimycotically active nail lacquer for the treatment or prophylaxis of fungal infections of nails and skin, comprising an optionally substituted 2-aminothiazole of formula (I) 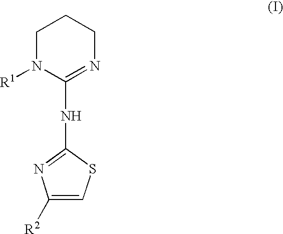
wherein
R¹ represents hydrogen or alkyl, and
R² represents a substituent of the formula 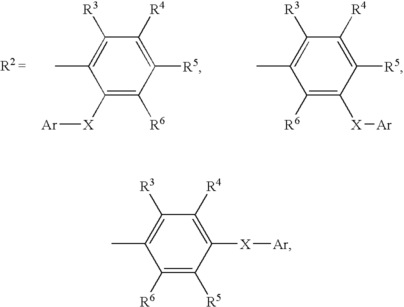
wherein
R³, R⁴, R⁵ and R⁶ independently from each other represent hydrogen, halogen, nitro, alkyl, alkoxy, alkoxy-carbonyl, dialkylamino, alkylthio, alkylsulphinyl, alkylsulphonyl, halogenoalkyl, halogenoalkoxy, halogenoalkylthio, halogenoalkylsulphinyl or halogenoalkylsulphonyl,
X represents oxygen, and
Ar represents an optionally substituted aryl moiety, and
their physiologically acceptable acid addition salts,
wherein the compounds of formula (I) are in equilibrium with the tautomeric compounds of formulae (Ia) and (Ib) 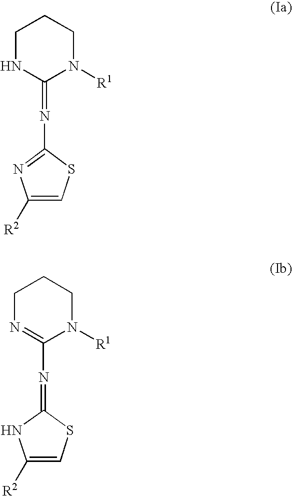
wherein R¹ and R² in each case have the abovementioned meanings.

3. A pharmaceutical, antimycotically active nail lacquer according to claim 0 or 0, characterized in that it comprises a permeation enhancer.

4. A pharmaceutical, antimycotically active nail lacquer according to claim 0, wherein the permeation enhancer is hyaluronate lyase.

5. A pharmaceutical, antimycotically active nail lacquer according to claim 1 or 2, characterized in that it comprises a polymeric film-forming agent.

6. A pharmaceutical, antimycotically active nail lacquer according to claim 0, characterized in that the film-forming agent belongs to the group of water-insoluble acrylate polymers or methacrylate polymers and comprises a copolymer that belongs to the compound class of alkylvinylethers, maleic acid anhydride, alkylated poly(vinylpyrrolidones) and ammonium methacrylates.

7. A pharmaceutical, antimycotically active nail lacquer according to claim 1 or 2, characterized in that it comprises a plasticizer.

8. A pharmaceutical, antimycotically active nail lacquer according to claim 1 or 2, characterized in that it comprises at least one solvent.

9. A pharmaceutical, antimycotically active nail lacquer according to claim 0, characterized in that it comprises as a solvent ethanol, ethyl acetate, butyl acetate, isopropanol, acetone, methyl ethyl ketone, triacetin, tripropionin, diethylene glycol monoethyl ether, isopropyl acetate, or a mixture of two or more of said solvents.

10. A pharmaceutical, antimycotically active nail lacquer according to claim 1 or 2, comprising
from 0.1 to 20% antimycotic agent,
from 0.5 to 35% permeation enhancer,
from 0.5 to 40% film-forming polymer,
from 5 to 80% volatile solvent, and
from 1.0 to 10% plasticizer.

11. A pharmaceutical, antimycotically active nail lacquer according to claim 1 or 2, wherein the antimycotically active ingredient is Abafungin.

12. A method for the treatment of fungal infections, in which a pharmaceutical, antimycotically active nail lacquer according to claim 1 or 2 is applied to infected nails.

13. A method for the prophylaxis of fungal infections, in which a pharmaceutical, antimycotically active nail lacquer according to claim 1 or 2 is applied to nails of subjects who are in danger of being affected by a nail fungal infection.
